(19) United States
(12) Patent Application Publication
(10) Pub. No.: US 2019/0022002 A1
(43) Pub. Date: Jan. 24, 2019

(54) LIPOSOME FORMULATION FOR DELIVERY OF WNT SIGNAL PATHWAY INHIBITOR AND A METHOD FOR PREPARING THE SAME

(71) Applicant: Curegenix, Inc., Guangzhou (CN)

(72) Inventors: Yuhong Xu, Guangzhou (CN); Meiqing Tu, Guangzhou (CN); Xiaojing Chen, Guangzhou (CN)

(21) Appl. No.: 15/944,568

(22) Filed: Apr. 3, 2018

(30) Foreign Application Priority Data
Jul. 21, 2017 (CN) 201710600117.7

(51) Int. Cl. A61K 9/127; A61K 31/444; A61K 47/46

(52) U.S. Cl. CPC A61K 9/127; A61K 9/0019; A61K 47/46; A61K 31/444

(57) ABSTRACT

A liposome formulation for delivery of Wnt signal pathway inhibitor is provided herein, which comprises lipid molecules and Wnt signal pathway inhibitor, wherein Wnt signal pathway inhibitor encapsulated within the liposome formulation has a concentration ranging from 0.07 mg/ml to 0.5 mg/ml.

### CROSS REFERENCE TO RELATED APPLICATION

This application claims the benefit of, and priority to, Chinese Application No.: 201710600117.7, filed on Jul. 21, 2017, the entire disclosure of which is hereby incorporated by reference in its entirety.

### FIELD OF THE INVENTION

The present invention relates to a liposome formulation for delivery of a Wnt signal pathway inhibitor and a method for preparing the same.

### BACKGROUND

The Wnt signal pathway involves a variety of complicated biochemical reactions, and plays a key role in regulation of embryonic development. Dysfunction of Wnt pathway has close correlation with tumorigenesis. Recently, the relation between the Wnt signal pathway and human tumors has attracted increasing attention and becomes a worldwide hot research topic. It has been shown that Wnt signal pathway inhibitor can be widely used in treatment of tumors. However, further clinical investigation of Wnt signal pathway inhibitors as a novel pharmaceutical is hindered due to the toxicity of Wnt signal pathway inhibitor.

A liposome is a microvesicle with a lipid bilayer which resembles the structure of a cell membrane and can be used as a superior vehicle for pharmaceuticals, with various advantages such as excellent biocompatibility, targeting ability, capability of increasing effective concentration of pharmaceuticals and reducing toxicity of pharmaceuticals and the like. Therefore, liposomes can be used as a delivery vehicle for Wnt signal pathway inhibitors in the treatment of cancers and tumors.

### SUMMARY

In one aspect, embodiments of the present invention provide a liposome formulation for delivery of a Wnt signal pathway inhibitor, comprising lipid molecules and the Wnt signal pathway inhibitor, wherein the Wnt signal pathway inhibitor encapsulated within the liposome formulation has a concentration ranging from 0.07 mg/ml to 0.5 mg/ml.

The Wnt signal pathway inhibitor within the liposome formulation as provided herein is selected from the compounds having the following Formula I:

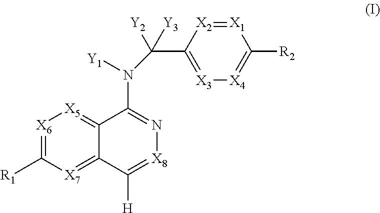

or a pharmaceutically acceptable salt thereof, wherein

X₁, X₂, X₃ and X₄ are independently CR₄ or N;

Y₁, Y₂, and Y₃ are independently hydrogen,

R₁ is

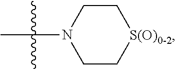

aryl, morpholinyl, piperazinyl, or 6 membered heteroaryl ring containing 1-2 heteroatoms selected from N, O and S, each of which can be optionally substituted with R₄;

R₂ is

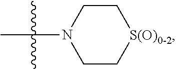

aryl, morpholinyl, piperazinyl, or 6 membered heteroaryl ring containing 1-2 heteroatoms selected from N, O and S, each of which can be optionally substituted with R₄;

R₄ is hydrogen, halo, C₁₋₆alkoxyl, C₁₋₆alkyl, each of which can be optionally substituted with halo, hydroxyl, alkoxyl and cyano;

the 6 membered heteroaryl is selected from:

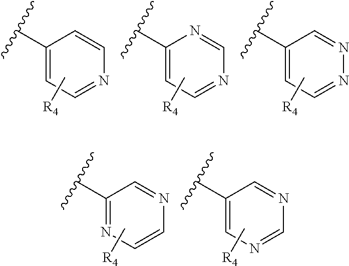

and wherein the Formula I has the following core structure:

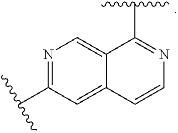 [SMILES: *c1cc2ccnc(*)c2cn1]

The Wnt signal pathway inhibitor within the liposome formulation as provided herein is selected from the compounds having the following Formula II:

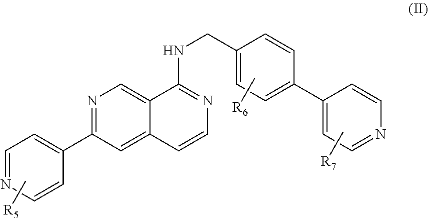

or a pharmaceutically acceptable salt thereof, wherein

R₅, R₆ and R₇ are independently selected from the group consisting of hydrogen, halo, C₁₋₆alkoxyl, C₁₋₆alkyl, wherein, each of C₁₋₆alkoxyl and C₁₋₆alkyl can be optionally substituted with halo, hydroxyl, alkoxyl or cyano.

The Wnt signal pathway inhibitor within the liposome formulation as provided herein is the compound selected from the below table or the pharmaceutically acceptable salt thereof.

TABLE 1

| Compound | Structure |
|---|---|
| 1 | 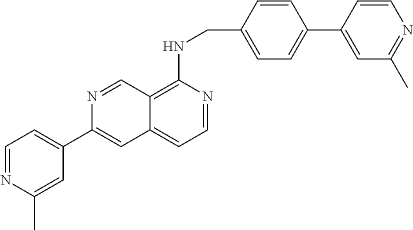 [SMILES: Cc1cc(-c2ccc(CNc3nccc4cc(-c5ccnc(C)c5)ncc34)cc2)ccn1] |
| 2 | 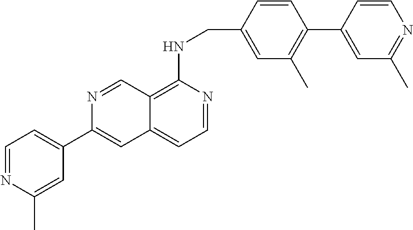 [SMILES: Cc1cc(-c2cc3ccnc(NCc4ccc(-c5ccnc(C)c5)c(C)c4)c3cn2)ccn1] |
| 3 | 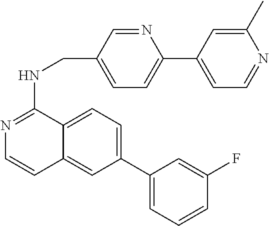 [SMILES: Cc1cc(-c2ccc(CNc3nccc4cc(-c5cccc(F)c5)ccc34)cn2)ccn1] |
| 4 | 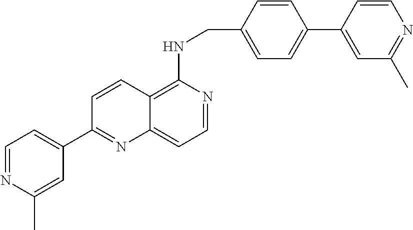 [SMILES: Cc1cc(-c2ccc(CNc3nccc4nc(-c5ccnc(C)c5)ccc34)cc2)ccn1] |
| 5 | 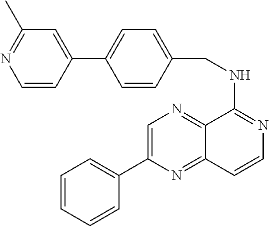 [SMILES: Cc1cc(-c2ccc(CNc3nccc4nc(-c5ccccc5)cnc34)cc2)ccn1] |
| 6 | 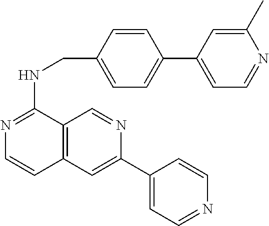 [SMILES: Cc1cc(-c2ccc(CNc3nccc4cc(-c5ccncc5)ncc34)cc2)ccn1] |
| 7 | 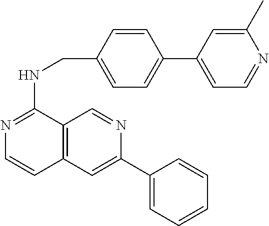 [SMILES: Cc1cc(-c2ccc(CNc3nccc4cc(-c5ccccc5)ncc34)cc2)ccn1] |
| 8 | 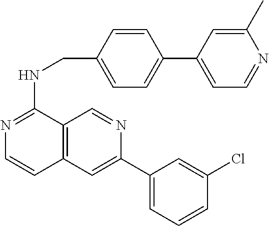 [SMILES: Cc1cc(-c2ccc(CNc3nccc4cc(-c5cccc(Cl)c5)ncc34)cc2)ccn1] |
| 9 | 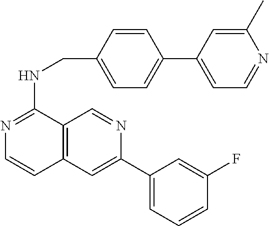 [SMILES: Cc1cc(-c2ccc(CNc3nccc4cc(-c5cccc(F)c5)ncc34)cc2)ccn1] |
| 10 | 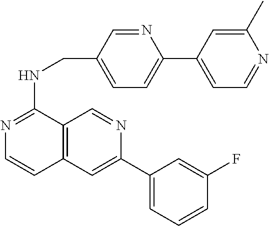 [SMILES: Cc1cc(-c2ccc(CNc3nccc4cc(-c5cccc(F)c5)ncc34)cn2)ccn1] |
| 11 | 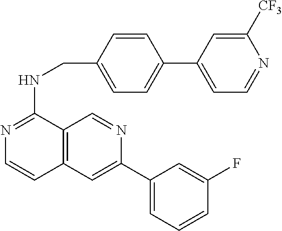 [SMILES: Fc1cccc(-c2cc3ccnc(NCc4ccc(-c5ccnc(C(F)(F)F)c5)cc4)c3cn2)c1] |
| 12 | 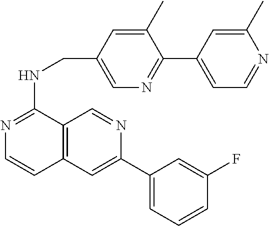 [SMILES: Cc1cc(-c2ncc(CNc3nccc4cc(-c5cccc(F)c5)ncc34)cc2C)ccn1] |
| 13 | 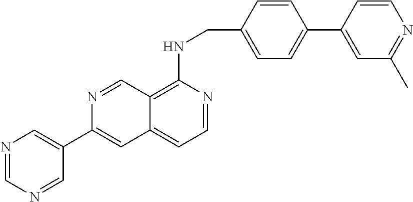 [SMILES: Cc1cc(-c2ccc(CNc3nccc4cc(-c5cncnc5)ncc34)cc2)ccn1] |
| 14 | 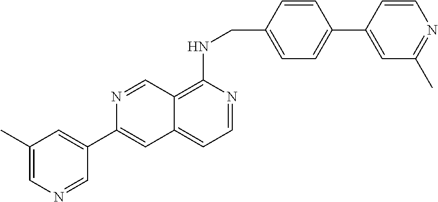 |
| 15 | 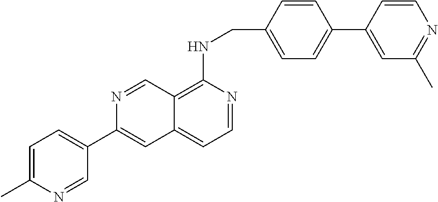 |
| 16 | 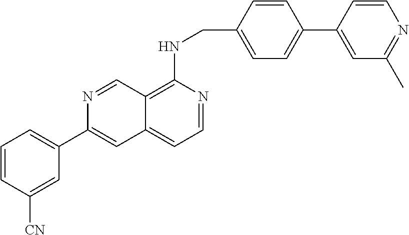 [SMILES: Cc1cc(-c2ccc(CNc3nccc4cc(-c5cccc(C#N)c5)ncc34)cc2)ccn1] |
| 17 | 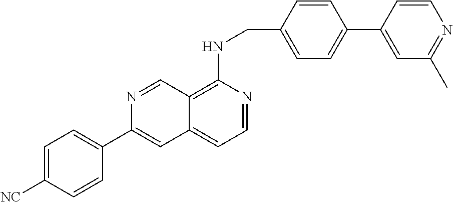 [SMILES: Cc1cc(-c2ccc(CNc3nccc4cc(-c5ccc(C#N)cc5)ncc34)cc2)ccn1] |
| 18 | 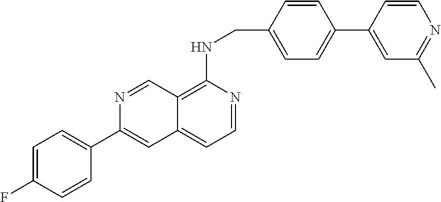 [SMILES: Cc1cc(-c2ccc(CNc3nccc4cc(-c5ccc(F)cc5)ncc34)cc2)ccn1] |
| 19 | 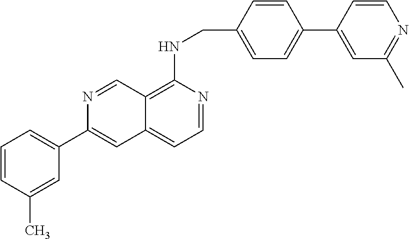 [SMILES: Cc1cccc(-c2cc3ccnc(NCc4ccc(-c5ccnc(C)c5)cc4)c3cn2)c1] |
| 20 | 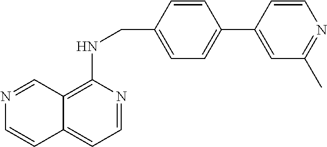 [SMILES: Cc1cc(-c2ccc(CNc3nccc4ccncc34)cc2)ccn1] |
| 21 | 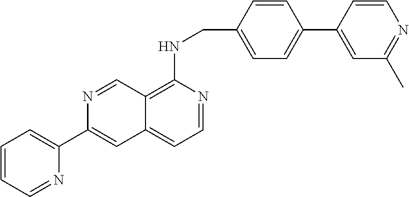 [SMILES: Cc1cc(-c2ccc(CNc3nccc4cc(-c5ccccn5)ncc34)cc2)ccn1] |
| 22 | 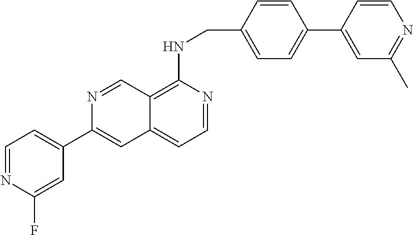 [SMILES: Cc1cc(-c2ccc(CNc3nccc4cc(-c5ccnc(F)c5)ncc34)cc2)ccn1] |
| 23 | 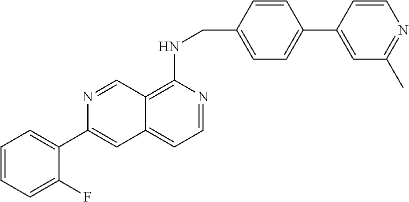 [SMILES: Cc1cc(-c2ccc(CNc3nccc4cc(-c5ccccc5F)ncc34)cc2)ccn1] |
| 24 | 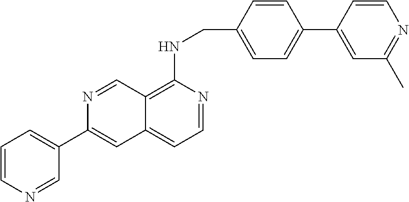 [SMILES: Cc1cc(-c2ccc(CNc3nccc4cc(-c5cccnc5)ncc34)cc2)ccn1] |
| 25 | 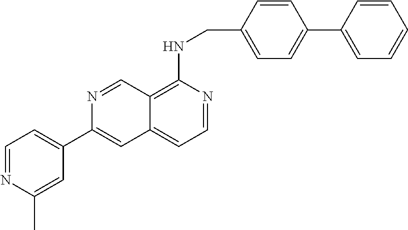 [SMILES: Cc1cc(-c2cc3ccnc(NCc4ccc(-c5ccccc5)cc4)c3cn2)ccn1] |
| 26 | 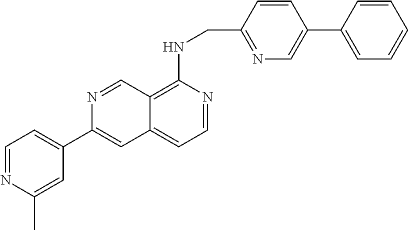 [SMILES: Cc1cc(-c2cc3ccnc(NCc4ccc(-c5ccccc5)cn4)c3cn2)ccn1] |
| 27 | 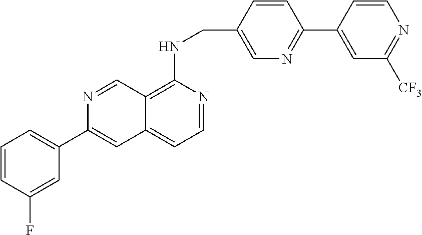 [SMILES: Fc1cccc(-c2cc3ccnc(NCc4ccc(-c5ccnc(C(F)(F)F)c5)nc4)c3cn2)c1] |
| 28 | 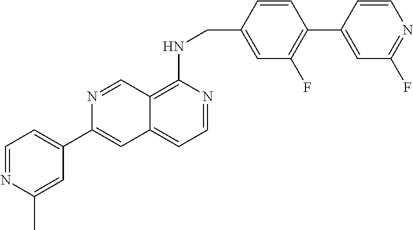 [SMILES: Cc1cc(-c2cc3ccnc(NCc4ccc(-c5ccnc(F)c5)c(F)c4)c3cn2)ccn1] |
| 29 | 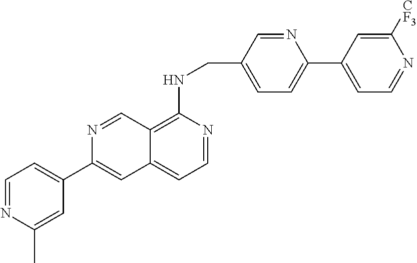 [SMILES: Cc1cc(-c2cc3ccnc(NCc4ccc(-c5ccnc(C(F)(F)F)c5)nc4)c3cn2)ccn1] |
| 30 | 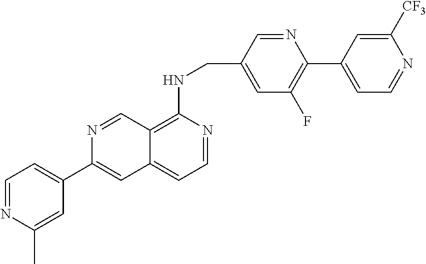 [SMILES: Cc1cc(-c2cc3ccnc(NCc4cnc(-c5ccnc(C(F)(F)F)c5)c(F)c4)c3cn2)ccn1] |
| 31 | 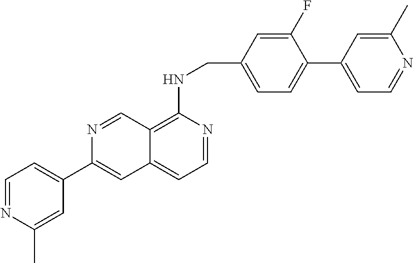 [SMILES: Cc1cc(-c2cc3ccnc(NCc4ccc(-c5ccnc(C)c5)c(F)c4)c3cn2)ccn1] |
| 32 | 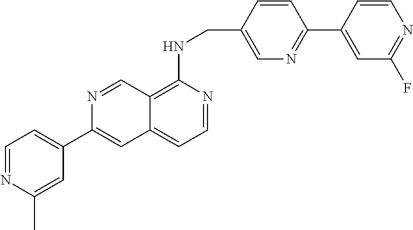 [SMILES: Cc1cc(-c2cc3ccnc(NCc4ccc(-c5ccnc(F)c5)nc4)c3cn2)ccn1] |
| 33 | 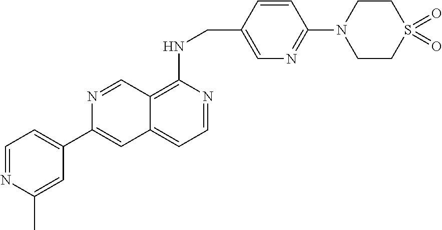 [SMILES: Cc1cc(-c2cc3ccnc(NCc4ccc(N5CCS(=O)(=O)CC5)nc4)c3cn2)ccn1] |
| 34 | 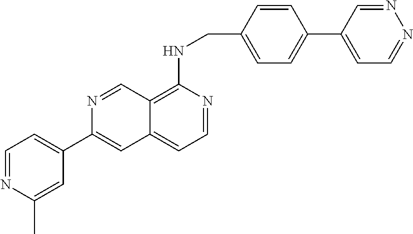 [SMILES: Cc1cc(-c2cc3ccnc(NCc4ccc(-c5ccnnc5)cc4)c3cn2)ccn1] |
| 35 | 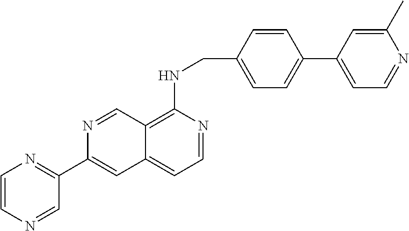 [SMILES: Cc1cc(-c2ccc(CNc3nccc4cc(-c5cnccn5)ncc34)cc2)ccn1] |
| 36 | 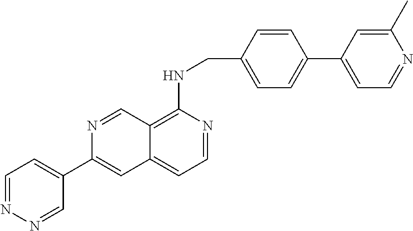 [SMILES: Cc1cc(-c2ccc(CNc3nccc4cc(-c5ccnnc5)ncc34)cc2)ccn1] |
| 37 | 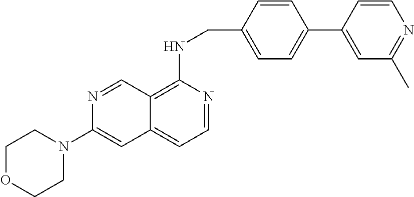 [SMILES: Cc1cc(-c2ccc(CNc3nccc4cc(N5CCOCC5)ncc34)cc2)ccn1] |
| 38 | 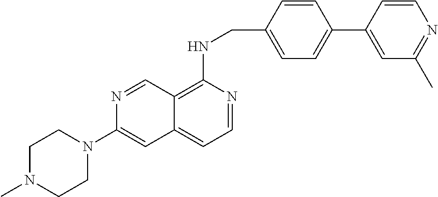 |
| 39 | 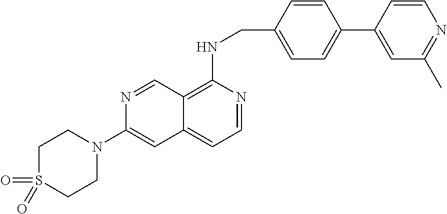 [SMILES: Cc1cc(-c2ccc(CNc3nccc4cc(N5CCS(=O)(=O)CC5)ncc34)cc2)ccn1] |
| 40 | 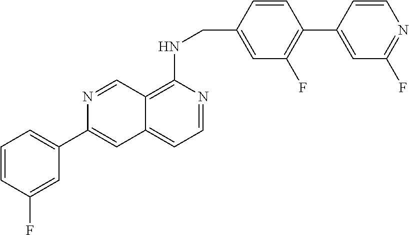 [SMILES: Fc1cccc(-c2cc3ccnc(NCc4ccc(-c5ccnc(F)c5)c(F)c4)c3cn2)c1] |
| 41 | 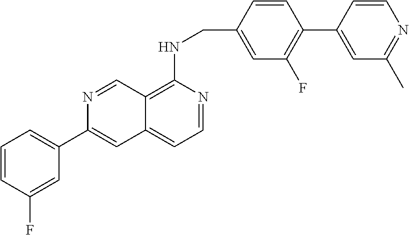 [SMILES: Cc1cc(-c2ccc(CNc3nccc4cc(-c5cccc(F)c5)ncc34)cc2F)ccn1] |
| 42 | 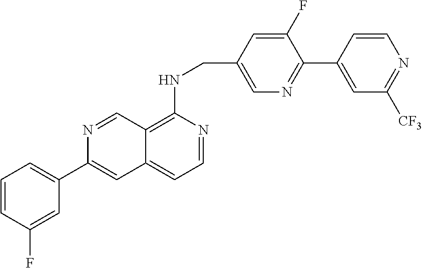 [SMILES: Fc1cccc(-c2cc3ccnc(NCc4cnc(-c5ccnc(C(F)(F)F)c5)c(F)c4)c3cn2)c1] |
| 43 | 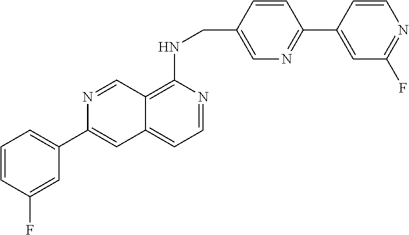 [SMILES: Fc1cccc(-c2cc3ccnc(NCc4ccc(-c5ccnc(F)c5)nc4)c3cn2)c1] |
| 44 | 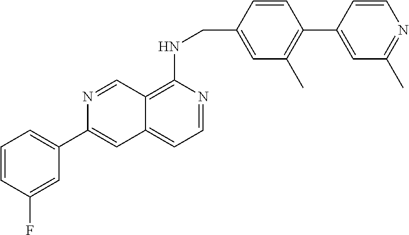 [SMILES: Cc1cc(-c2ccc(CNc3nccc4cc(-c5cccc(F)c5)ncc34)cc2C)ccn1] |
| 45 | 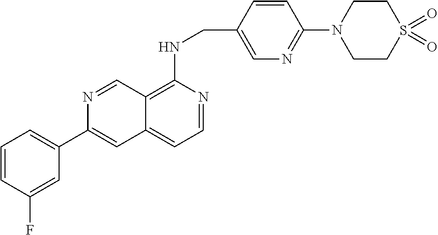 [SMILES: O=S1(=O)CCN(c2ccc(CNc3nccc4cc(-c5cccc(F)c5)ncc34)cn2)CC1] |
| 46 | 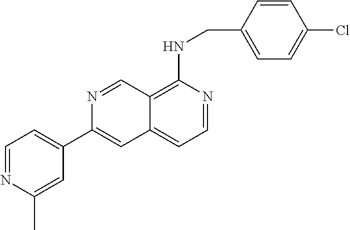 [SMILES: Cc1cc(-c2cc3ccnc(NCc4ccc(Cl)cc4)c3cn2)ccn1] |
| 47 | 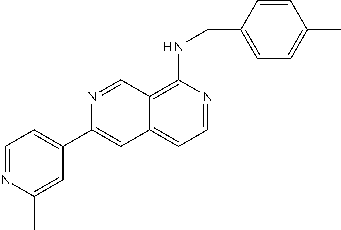 [SMILES: Cc1ccc(CNc2nccc3cc(-c4ccnc(C)c4)ncc23)cc1] |
| 48 | 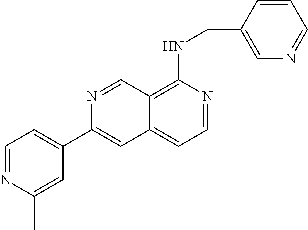 [SMILES: Cc1cc(-c2cc3ccnc(NCc4cccnc4)c3cn2)ccn1] |
| 49 | 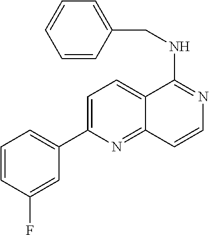 [SMILES: Fc1cccc(-c2ccc3c(NCc4ccccc4)nccc3n2)c1] |
| 50 | 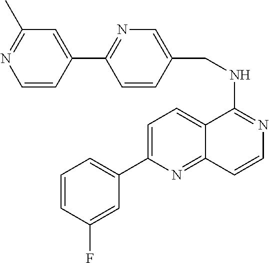 [SMILES: Cc1cc(-c2ccc(CNc3nccc4nc(-c5cccc(F)c5)ccc34)cn2)ccn1] |
| 51 | 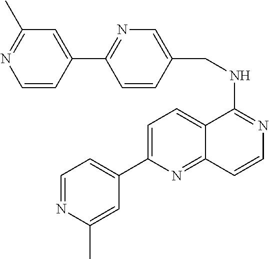 [SMILES: Cc1cc(-c2ccc(CNc3nccc4nc(-c5ccnc(C)c5)ccc34)cn2)ccn1] |
| 52 | 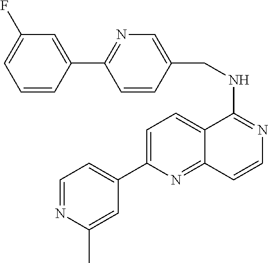 [SMILES: Cc1cc(-c2ccc3c(NCc4ccc(-c5cccc(F)c5)nc4)nccc3n2)ccn1] |
| 53 | 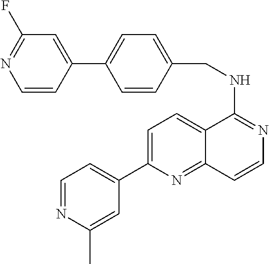 [SMILES: Cc1cc(-c2ccc3c(NCc4ccc(-c5ccnc(F)c5)cc4)nccc3n2)ccn1] |
| 54 | 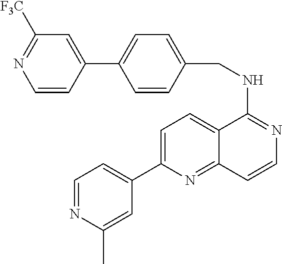 [SMILES: Cc1cc(-c2ccc3c(NCc4ccc(-c5ccnc(C(F)(F)F)c5)cc4)nccc3n2)ccn1] |
| 55 | 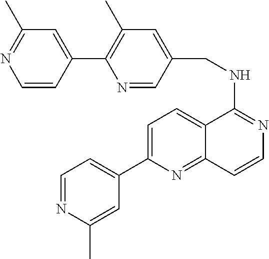 [SMILES: Cc1cc(-c2ccc3c(NCc4cnc(-c5ccnc(C)c5)c(C)c4)nccc3n2)ccn1] |
| 56 | 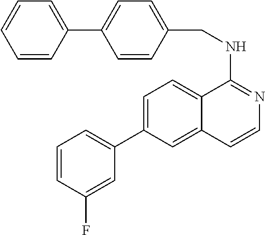 [SMILES: Fc1cccc(-c2ccc3c(NCc4ccc(-c5ccccc5)cc4)nccc3c2)c1] |
| 57 | 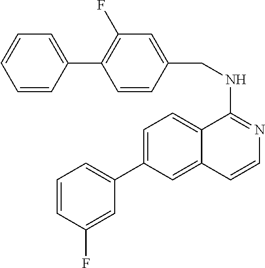 [SMILES: Fc1cccc(-c2ccc3c(NCc4ccc(-c5ccccc5)c(F)c4)nccc3c2)c1] |
| 58 | 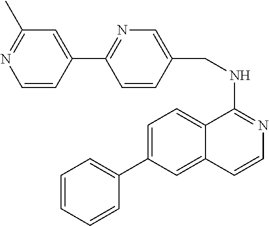 [SMILES: Cc1cc(-c2ccc(CNc3nccc4cc(-c5ccccc5)ccc34)cn2)ccn1] |
| 59 | 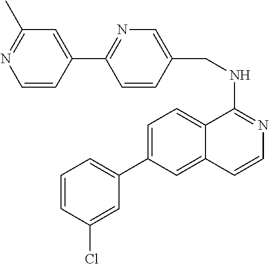 [SMILES: Cc1cc(-c2ccc(CNc3nccc4cc(-c5cccc(Cl)c5)ccc34)cn2)ccn1] |
| 60 | 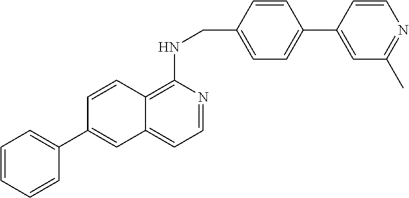 [SMILES: Cc1cc(-c2ccc(CNc3nccc4cc(-c5ccccc5)ccc34)cc2)ccn1] |
| 61 | 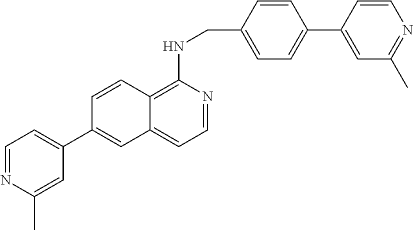 [SMILES: Cc1cc(-c2ccc(CNc3nccc4cc(-c5ccnc(C)c5)ccc34)cc2)ccn1] |
| 62 | 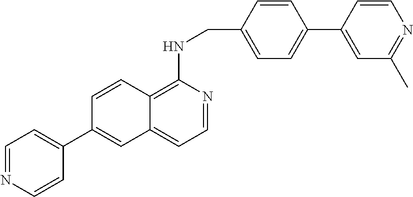 [SMILES: Cc1cc(-c2ccc(CNc3nccc4cc(-c5ccncc5)ccc34)cc2)ccn1] |
| 63 | 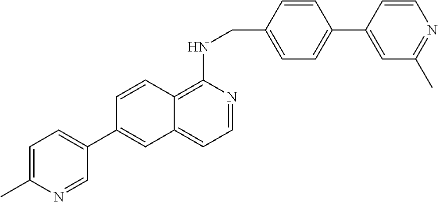 |
| 64 | 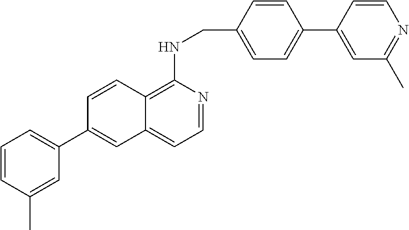 [SMILES: Cc1cccc(-c2ccc3c(NCc4ccc(-c5ccnc(C)c5)cc4)nccc3c2)c1] |
| 65 | 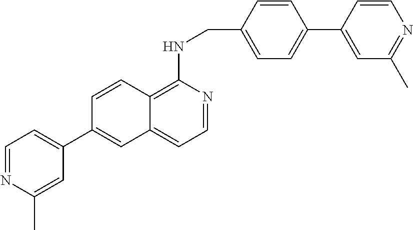 [SMILES: Cc1cc(-c2ccc(CNc3nccc4cc(-c5ccnc(C)c5)ccc34)cc2)ccn1] |
| 66 | 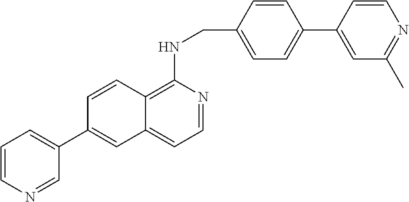 [SMILES: Cc1cc(-c2ccc(CNc3nccc4cc(-c5cccnc5)ccc34)cc2)ccn1] |
| 67 | 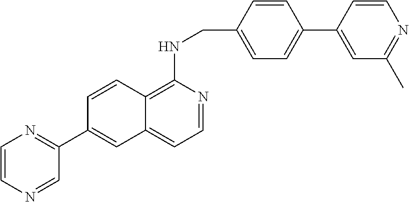 [SMILES: Cc1cc(-c2ccc(CNc3nccc4cc(-c5cnccn5)ccc34)cc2)ccn1] |
| 68 | 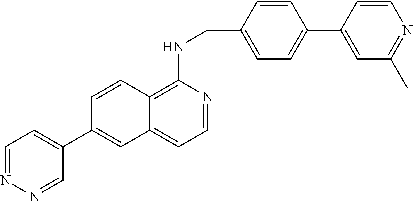 [SMILES: Cc1cc(-c2ccc(CNc3nccc4cc(-c5ccnnc5)ccc34)cc2)ccn1] |
| 69 | 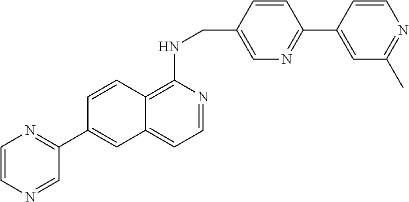 [SMILES: Cc1cc(-c2ccc(CNc3nccc4cc(-c5cnccn5)ccc34)cn2)ccn1] |
| 70 | 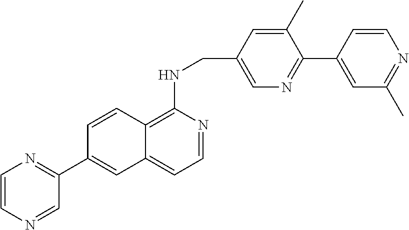 [SMILES: Cc1cc(-c2ncc(CNc3nccc4cc(-c5cnccn5)ccc34)cc2C)ccn1] |
| 71 | 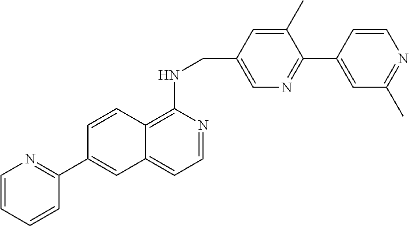 [SMILES: Cc1cc(-c2ncc(CNc3nccc4cc(-c5ccccn5)ccc34)cc2C)ccn1] |
| 72 | 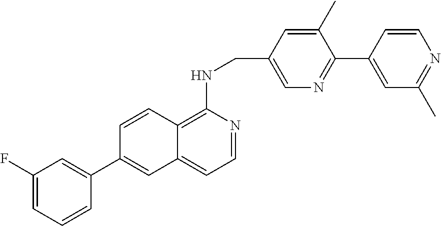 [SMILES: Cc1cc(-c2ncc(CNc3nccc4cc(-c5cccc(F)c5)ccc34)cc2C)ccn1] |
| 73 | 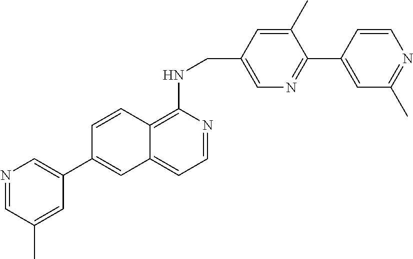 [SMILES: Cc1cncc(-c2ccc3c(NCc4cnc(-c5ccnc(C)c5)c(C)c4)nccc3c2)c1] |
| 74 | 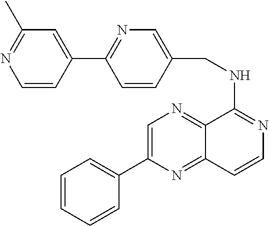 [SMILES: Cc1cc(-c2ccc(CNc3nccc4nc(-c5ccccc5)cnc34)cn2)ccn1] |
| 75 | 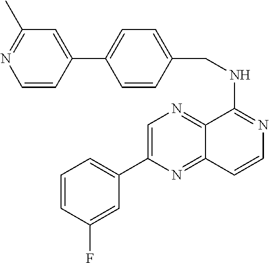 [SMILES: Cc1cc(-c2ccc(CNc3nccc4nc(-c5cccc(F)c5)cnc34)cc2)ccn1] |
| 76 | 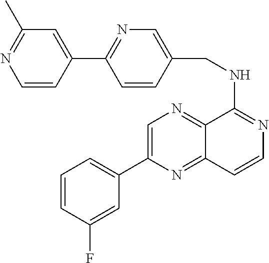 [SMILES: Cc1cc(-c2ccc(CNc3nccc4nc(-c5cccc(F)c5)cnc34)cn2)ccn1] |
| 77 | 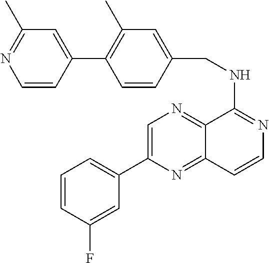 [SMILES: Cc1cc(-c2ccc(CNc3nccc4nc(-c5cccc(F)c5)cnc34)cc2C)ccn1] |
| 78 | 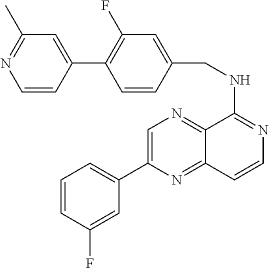 [SMILES: Cc1cc(-c2ccc(CNc3nccc4nc(-c5cccc(F)c5)cnc34)cc2F)ccn1] |
| 79 | 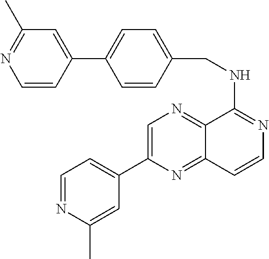 [SMILES: Cc1cc(-c2ccc(CNc3nccc4nc(-c5ccnc(C)c5)cnc34)cc2)ccn1] |
| 80 | 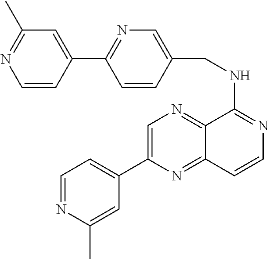 [SMILES: Cc1cc(-c2ccc(CNc3nccc4nc(-c5ccnc(C)c5)cnc34)cn2)ccn1] |
| 81 | 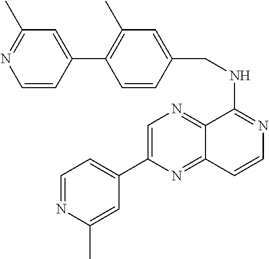 [SMILES: Cc1cc(-c2cnc3c(NCc4ccc(-c5ccnc(C)c5)c(C)c4)nccc3n2)ccn1] |
| 82 | 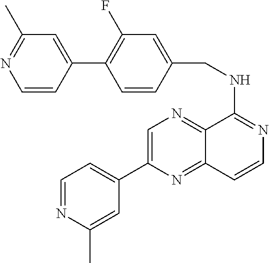 [SMILES: Cc1cc(-c2cnc3c(NCc4ccc(-c5ccnc(C)c5)c(F)c4)nccc3n2)ccn1] |
| 83 | 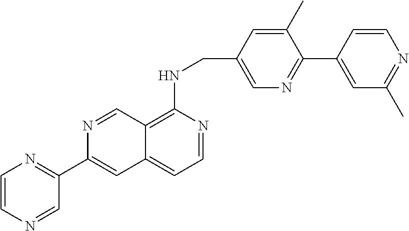 [SMILES: Cc1cc(-c2ncc(CNc3nccc4cc(-c5cnccn5)ncc34)cc2C)ccn1] |
| 84 | 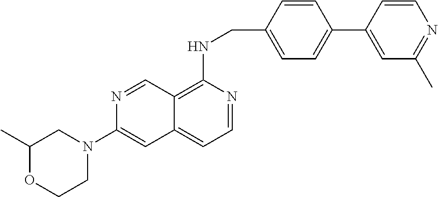 |
| 85 | 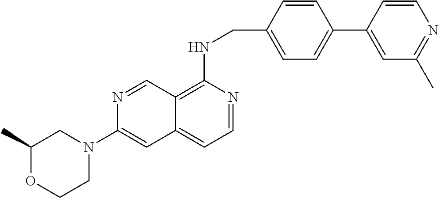 |
| 86 | 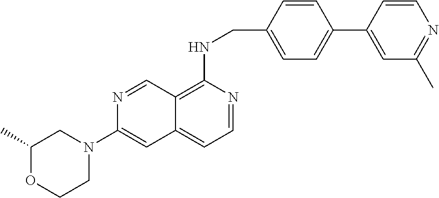 |
| 87 | 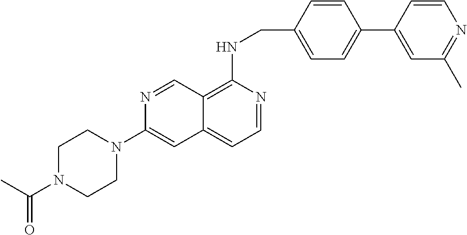 [SMILES: CC(=O)N1CCN(c2cc3ccnc(NCc4ccc(-c5ccnc(C)c5)cc4)c3cn2)CC1] |
| 88 | 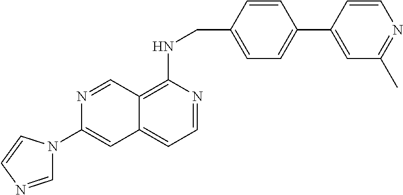 [SMILES: Cc1cc(-c2ccc(CNc3nccc4cc(-n5ccnc5)ncc34)cc2)ccn1] |
| 89 | 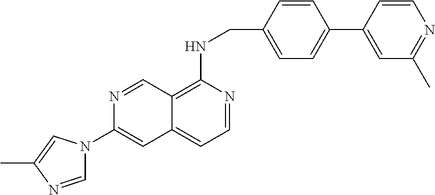 |
| 90 | 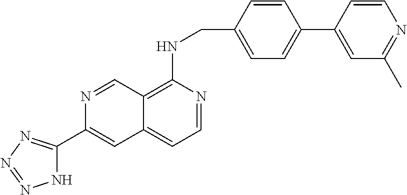 [SMILES: Cc1cc(-c2ccc(CNc3nccc4cc(-c5nnn[nH]5)ncc34)cc2)ccn1] |
| 91 | 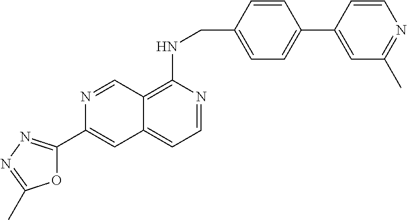 [SMILES: Cc1cc(-c2ccc(CNc3nccc4cc(-c5nnc(C)o5)ncc34)cc2)ccn1] |
| 92 | 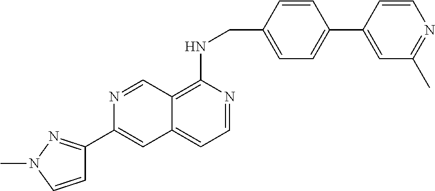 |
| 93 | 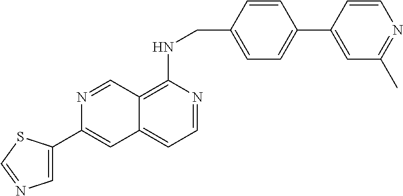 [SMILES: Cc1cc(-c2ccc(CNc3nccc4cc(-c5cncs5)ncc34)cc2)ccn1] |
| 94 | 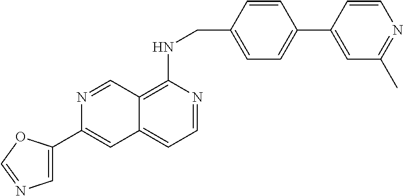 [SMILES: Cc1cc(-c2ccc(CNc3nccc4cc(-c5cnco5)ncc34)cc2)ccn1] |
| 95 | 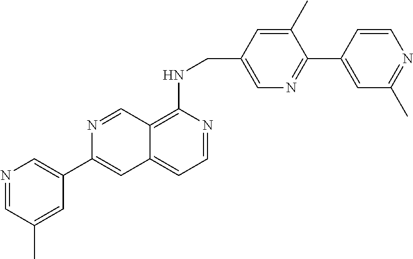 [SMILES: Cc1cncc(-c2cc3ccnc(NCc4cnc(-c5ccnc(C)c5)c(C)c4)c3cn2)c1] |
| 96 | 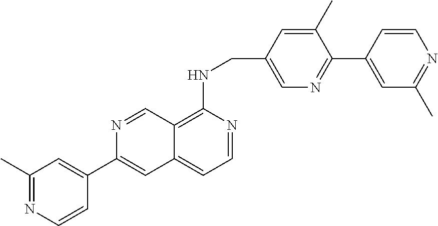 |
| 97 | 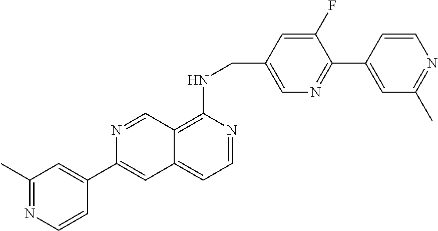 |
| 98 | 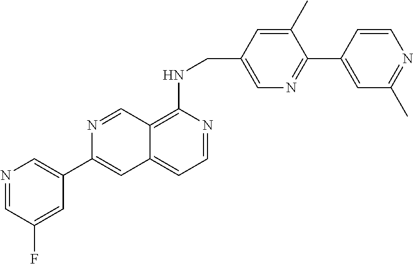 [SMILES: Cc1cc(-c2ncc(CNc3nccc4cc(-c5cncc(F)c5)ncc34)cc2C)ccn1] |
| 99 | 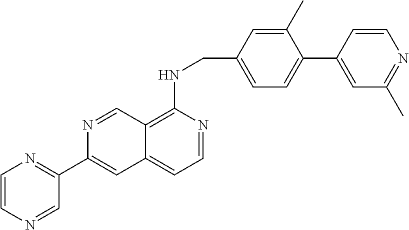 [SMILES: Cc1cc(-c2ccc(CNc3nccc4cc(-c5cnccn5)ncc34)cc2C)ccn1] |
| 100 | 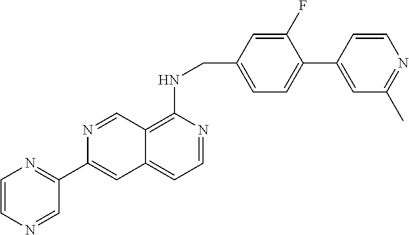 [SMILES: Cc1cc(-c2ccc(CNc3nccc4cc(-c5cnccn5)ncc34)cc2F)ccn1] |
| 101 | 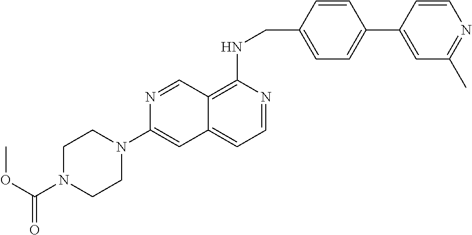 [SMILES: COC(=O)N1CCN(c2cc3ccnc(NCc4ccc(-c5ccnc(C)c5)cc4)c3cn2)CC1] |
| 102 | 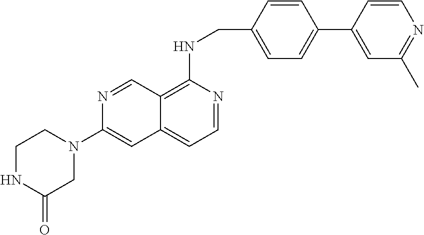 [SMILES: Cc1cc(-c2ccc(CNc3nccc4cc(N5CCNC(=O)C5)ncc34)cc2)ccn1] |
| 103 | 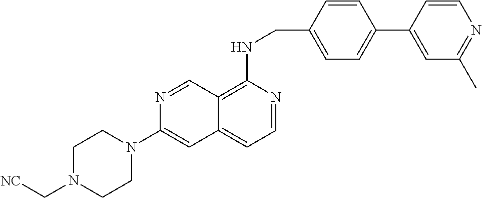 [SMILES: Cc1cc(-c2ccc(CNc3nccc4cc(N5CCN(CC#N)CC5)ncc34)cc2)ccn1] |
| 104 | 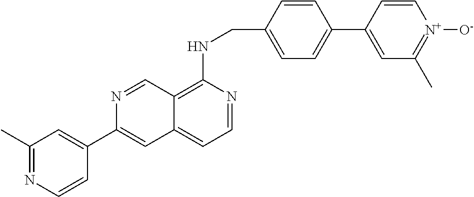 [SMILES: Cc1cc(-c2cc3ccnc(NCc4ccc(-c5cc[n+]([O-])c(C)c5)cc4)c3cn2)ccn1] |
| 105 | 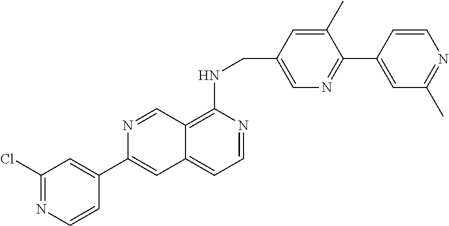 [SMILES: Cc1cc(-c2ncc(CNc3nccc4cc(-c5ccnc(Cl)c5)ncc34)cc2C)ccn1] |
| 106 | 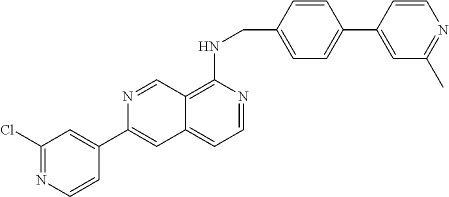 [SMILES: Cc1cc(-c2ccc(CNc3nccc4cc(-c5ccnc(Cl)c5)ncc34)cc2)ccn1] |
| 107 | 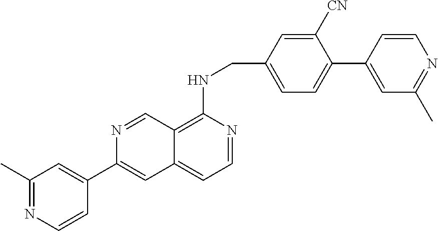 |
| 108 | 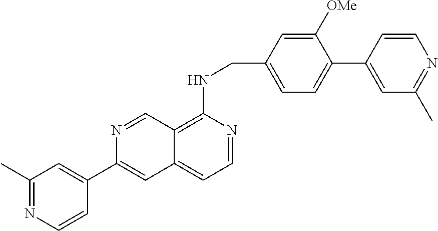 |
| 109 | 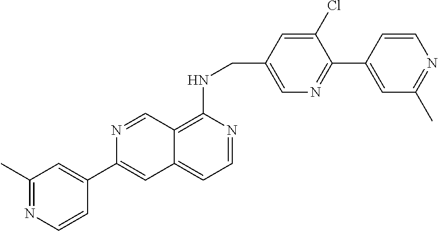 |
| 110 | 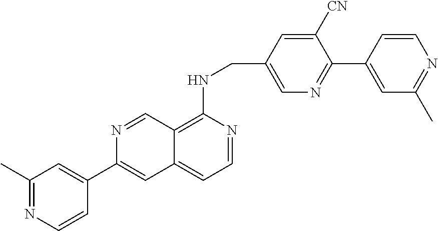 |
| 111 | 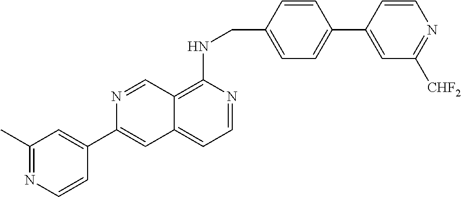 [SMILES: Cc1cc(-c2cc3ccnc(NCc4ccc(-c5ccnc(C(F)F)c5)cc4)c3cn2)ccn1] |

The lipid molecules in the liposome formulation as provided herein are soybean lecithin and cholesterol. The molar ratio between the lipid molecules and the Wnt signal pathway inhibitor ranges from 60:1 to 50:1. The molar ratio between soybean lecithin and cholesterol ranges from 20:1 to 10:1. The Wnt signal pathway inhibitor encapsulated within the liposome formulation has a concentration between 0.07 mg/ml and 0.4 mg/ml, or 0.07 mg/ml and 0.3 mg/ml, or 0.07 mg/ml and 0.2 mg/ml, or 0.07 mg/ml and 0.1 mg/ml.

The liposome formulation as provided herein has an average particle size of from 50 nm to 200 nm.

The liposome formulation as provided herein can be formulated as an oral formulation, or a subcutaneous injection formulation, or an intravenous injection formulation.

In another aspect, a method for preparing the liposome formulation as described above is provided herein, which comprises:

(1) providing an aqueous solution of a Wnt signal pathway inhibitor and providing an alcoholic solution of the lipid molecules,

(2) mixing the alcoholic solution of the lipid molecules and the aqueous solution of the Wnt signal pathway inhibitor,

(3) removing the alcoholic solvent to form the liposome formulation with the Wnt signal pathway inhibitor encapsulated therein.

In yet another aspect, embodiments of the present invention relate to use of the liposome formulation as mentioned above in manufacturing a medicament for treating cancers.

### BRIEF DESCRIPTION OF THE DRAWINGS

FIG. 1 depicts a Cryo-TEM image of the liposome formulation according to Example 1 provided herein.

### DETAILED DESCRIPTION

### Example 1

The Liposome Formulation for Delivery of a Wnt Signal Pathway Inhibitor and the Method for Preparing the Same

The present example illustrates the liposome formulation for delivery of Wnt signal pathway inhibitor and the method for preparing the same, taking compound 28 as an example. The method for preparing the liposome formulation comprises:
(i) forming an alcoholic solution of the lipid molecules, comprising dissolving soybean lecithin and cholesterol in ethanol at a molar ratio of 20:1 to form the alcoholic solution of the lipid molecules;
(ii) forming ethanol solution of a Wnt signal pathway inhibitor at a concentration of 1 mg/ml;
(iii) sufficiently mixing the alcoholic solution of the lipid molecules and the ethanol solution of the Wnt signal pathway inhibitor (the molar ratio between Wnt signal pathway inhibitor and the sum amount of soybean lecithin and cholesterol is 1:60), and warming up to 50° C. in a water bath, and then adding phosphate buffers saline (PBS) solution to the ethanol solution (the volumetric ratio between the ethanol solution and the PBS solution is 1.5:8.5), thereby obtaining the liposome formulation (sample #1) of liposomes with the Wnt signal pathway inhibitor encapsulated therein.

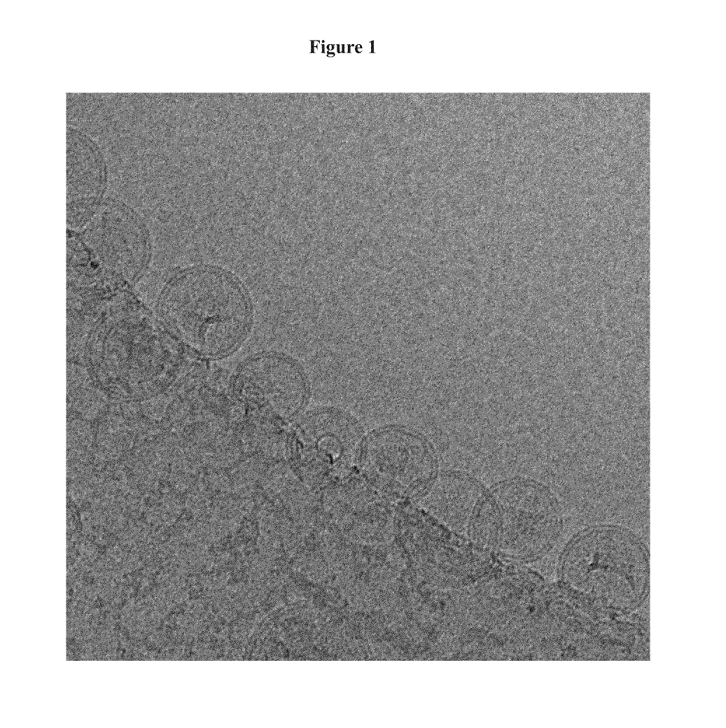

The Cryo-TME image of the liposome formulation, prepared according to the above method, is shown in FIG. 1. As shown in FIG. 1, the outer shell of the liposome formulation is formed from the lipid molecules and the Wnt signal pathway inhibitor was encapsulated within the liposome.

The liposome formulation had an average particle size of 96.5 nm as measured by using laser particle analyser (Malvern Corp.) according to light scattering principle.

Parameters for measuring the particle size include: 25° C., viscosity of 0.089 cP, reflex angle of 1.33, angle of 90 degree, balance for 60 seconds. The result was an average for 3 independent measurements.

The liposome formulation with a Wnt signal pathway inhibitor encapsulated therein at a certain concentration as provided herein is formed by mixing the specific lipid molecules and Wnt signal pathway inhibitor at a specific ratio therebetween. The toxicity of Wnt signal pathway inhibitor is reduced by encapsulating it within the liposome and a Wnt signal pathway inhibitor can be effectively delivered to the tumor tissue via the liposome formulation, so as to enhance anti-tumor effect.

What we claimed is:

1. A liposome formulation for delivery of a Wnt signal pathway inhibitor, comprising lipid molecules formed into liposomes and a Wnt signal pathway inhibitor, wherein said Wnt signal pathway inhibitor is encapsulated within the liposomes and the formulation has a concentration ranging from 0.07 mg/ml to 0.5 mg/ml of the Wnt signal inhibitor.

2. The liposome formulation of claim 1, wherein said Wnt signal pathway inhibitor has the following Formula I: 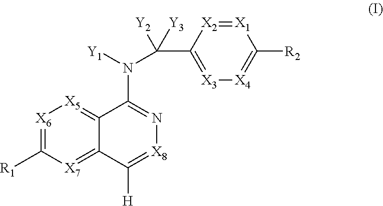
or a pharmaceutically acceptable salt thereof,
wherein
X₁, X₂, X₃ and X₄ are independently CR₄ or N;
Y₁, Y₂, and Y₃ are independently hydrogen,
R₁ is 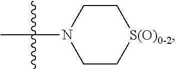
aryl, morpholinyl, piperazinyl, or 6 membered heteroaryl containing 1-2 heteroatoms selected from N, O and S, each of which can be optionally substituted with R₄;
R₂ is 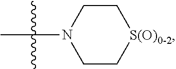
aryl, morpholinyl, piperazinyl, or 6 membered heteroaryl containing 1-2 heteroatoms selected from N, O and S, each of which can be optionally substituted with R₄;
R₄ is hydrogen, halo, C₁₋₆alkoxyl, C₁₋₆alkyl, each of which can be optionally substituted with halo, hydroxyl, alkoxyl and cyano;
the 6 membered heteroaryl is selected from: 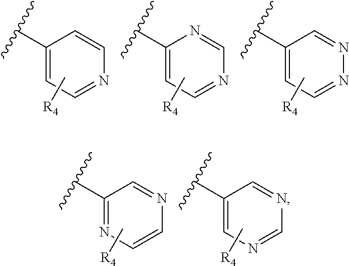
and wherein the Formula I has the following core structure:

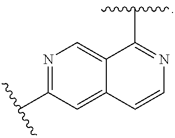 [SMILES: *c1cc2ccnc(*)c2cn1]

3. The liposome formulation of claim 2, wherein the Wnt signal pathway inhibitor is selected from the compounds having the following Formula II: 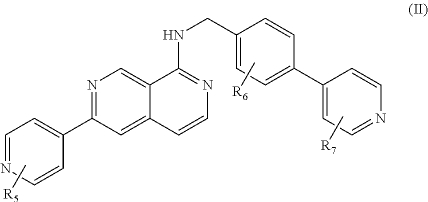
or a pharmaceutically acceptable salt thereof, wherein
R₅, R₆ and R₇ are independently selected from the group consisting of hydrogen, halo, C₁₋₆alkoxyl, C₁₋₆alkyl,
wherein, each of C₁₋₆alkoxyl and C₁₋₆alkyl can be optionally substituted with halo, hydroxyl, alkoxyl or cyano.

4. The liposome formulation of claim 2, wherein the Wnt signal pathway inhibitor is a compound selected from Table 1, or the pharmaceutically acceptable salt thereof.

5. The liposome formulation of any one of claim 1, wherein the lipid molecules are soybean lecithin and cholesterol.

6. The liposome formulation of claim 1, wherein the molar ratio between the lipid molecules and the Wnt signal pathway inhibitor ranges from 60:1 to 50:1.

7. The liposome formulation of claim 5, wherein the molar ratio between soybean lecithin and cholesterol ranges from 20:1 to 10:1.

8. The liposome formulation of claim 1, wherein Wnt signal pathway inhibitor encapsulated within the liposome formulation has a concentration between 0.07 mg/ml and 0.4 mg/ml, or 0.07 mg/ml and 0.3 mg/ml, or 0.07 mg/ml and 0.2 mg/ml, or 0.07 mg/ml and 0.1 mg/ml.

9. The liposome formulation of claim 1, wherein the liposome formulation has an average particle size of from 50 nm to 200 nm.

10. The liposome formulation of claim 1, wherein the liposome formulation is an oral formulation, or a subcutaneous injection formulation, or an intravenous injection formulation.
